(19) United States
(12) Patent Application Publication
(10) Pub. No.: US 2005/0177089 A1
(43) Pub. Date: Aug. 11, 2005

(54) Automatic pressurized liquid transfusion apparatus

(76) Inventor: George Lee, Taipei (TW)

Correspondence Address: Yi-Wen Tseng, 4331 Stevens Battle Lane, Fairfax, VA, 22033, US

(21) Appl. No.: 10/774,604

(22) Filed: Feb. 10, 2004

(51) Int. Cl. A61M 5/00

(52) U.S. Cl. 604/7

(57) ABSTRACT

An automatic liquid or blood transfusion system includes a pressurization device to supply a pressurized gas to at least one transfusion bag, and a gas reservoir connected to the pressurization device and a gas supply source. The pressurization device has an enclosure having a front panel and an open rear end, and inside the enclosure, the following devices are installed. A gas inlet is in communication with the gas reservoir. One end of a valve is in fluid communication with the gas inlet, and the other end of the valve is connected to a gas outlet. The gas outlet is connected to at least one liquid transfusion bag. A pressure gauge is installed at the gas outlet to monitor pressure of the gas flowing through the gas outlet. A pressure regulator is in fluid communication with the gas outlet to regulate pressure of the gas flowing through the gas outlet at a constant value. A control knob is installed at the front panel of the enclosure to set up the constant value. A lid is used to cover the rear open end of the enclosure, wherein the lid is perforated with a central opening allowing the gas outlet open at the rear end of the enclosure. The gas reservoir has a hollow shell with a gas outlet to be connected with the gas inlet of the pressurization device, a gas inlet to be connected to a gas supply source, and a flat rear panel with two lateral protruding sides. The system further has a wall mount having two slide channels for the protruding sides of the flat rear panel to slide through, and a bottom rim to hold the gas reservoir in the wall mount.

### BACKGROUND OF THE INVENTION

The present invention relates to an automatic pressurized liquid transfusion apparatus, and more particular, to an apparatus which automatically performs liquid transfusion to substitute the conventional manual liquid transfusion, so as to ensure the stability of liquid contained in the pressurized liquid transfusion bag and the safety of the patient.

The liquid transfusion bag is an essential medical application for hospitalized patients. The liquid transfusion is typically wrapped within a pressurized liquid transfusion bag to supply liquid or blood to the patient. How to minimize the attendance of the care giver and obtain a stable flow rate of the liquid or blood, so as to ensure the safety of the patient are currently the most important objectives to be achieved.

FIG. 1 illustrates a conventional pressurized liquid transfusion bag 1 connected under a separate pressure gauge 2. A connecting tube 3 is interconnected between a proper position of the liquid transfusion bag 1 and one end of a gas supply tube 4. The other end of the gas supply tube 4 is connected to a gas supply connector 51 of a liquid pressurization device 5. When a patient is in need of liquid or blood transfusion, a bag filled with the required liquid or blood is installed in the pressurized liquid transfusion bag 1. The pressurized liquid transfusion bag 1 and the pressure gauge 2 are then hung on a post or a frame. The care giver then connects the gas supply tube 4 with the gas supply connector 51 of the liquid pressurization device 5. Thereby, the care giver can press or squeeze the liquid pressurization device 5 to apply pressure to the liquid transfusion bag 1, such that the required liquid or blood contained in the liquid transfusion bag 1 is pressurized to flow into the patient.

The conventional manual liquid transfusion device as described above has the following drawbacks.

1. The manual pressure application can hardly control the gas pressure applied to the liquid transfusion bag, such that the flow rate of the liquid is unstable. The instability of liquid supply may even endanger the patient.

2. When the gas supply is insufficient, the flow of the liquid transfused to the patient will be either slow or stuck. The care giver has to press the liquid pressurization device 5 again to resume the proper flow of the liquid or blood routinely. It is thus very inconvenient and causes great human resource.

3. The manual liquid pressurization device is typically detached from the liquid transfusion system after the liquid starts to transfuse into the patient. When a pressurization step is required, the liquid pressurization device is connected to the liquid transfusion system again. In addition to the inconvenience in application, the system is easily damaged due to the frequent detachment and attachment.

It is therefore a substantially need to develop fixed automatic pressurized liquid transfusion device to overcome the drawbacks of the conventional pressurized liquid transfusion device.

### BRIEF SUMMARY OF THE INVENTION

The present invention provides an automatic liquid or blood transfusion system, including a pressurization device to supply a pressurized gas to at least one transfusion bag, and a gas reservoir connected to the pressurization device and a gas supply source. The pressurization device includes an enclosure having a front panel and an open rear end, and inside the enclosure, the following devices are installed. A gas inlet is in communication with the gas reservoir. One end of a valve is in fluid communication with the gas inlet, and the other end of the valve is connected to a gas outlet. The gas outlet is connected to at least one liquid transfusion bag. A pressure gauge is installed at the gas outlet to monitor pressure of the gas flowing through the gas outlet. A pressure regulator is in fluid communication with the gas outlet to regulate pressure of the gas flowing through the gas outlet at a constant value. A control knob is installed at the front panel of the enclosure to set up the constant value. A lid is used to cover the rear open end of the enclosure, wherein the lid is perforated with a central opening allowing the gas outlet open at the rear end of the enclosure. The gas reservoir has a hollow shell with a gas outlet to be connected with the gas inlet of the pressurization device, a gas inlet to be connected to a gas supply source, and a flat rear panel with two lateral protruding sides. The system further comprises a wall mount having two slide channels for the protruding sides of the flat rear panel to slide through, and a bottom rim to hold the gas reservoir in the wall mount.

Preferably, a switch is installed at the gas outlet of the pressurization device. The system further comprises the gas delivery tube with one proximal end connected to the gas outlet of the pressurization device and one distal end connected to the liquid transfusion bag. A branching device is connected to the distal end of the gas delivery tube allowing the pressurization device to be connected to more than one liquid transfusion bag. The branching device includes a Y-piece, for example. The system further comprises a gas supply tube having a proximal end connected to the gas inlet of the gas reservoir and a distal end connected to the gas supply source. The gas supply tube further comprises a plug installed at the distal end of the gas supply tube and a switch installed at the plug. A gas meter is further mounted on top of the gas transfusion bag and connected to the gas supply source. The system may further comprise a turning knob to adjust gas leakage ratio of the pressure regulator.

### BRIEF DESCRIPTION OF THE DRAWINGS

These, as well as other features of the present invention, will become apparent upon reference to the drawings wherein:

FIG. 1 shows a conventional liquid or blood transfusion system;

FIG. 2 shows an exploded view of a pressurization device for liquid or blood transfusion provided by the present invention;

FIG. 3 shows a perspective view of a liquid/blood transfusion system provided by the present invention;

FIG. 4 shows a rear panel of the pressurization device of the liquid/blood transfusion system as shown in FIGS. 2 and 3;

FIG. 5 shows a front panel of the pressurization device in one operation mode;

FIG. 6 shows the front panel of the pressurization device in another operation mode; and

FIG. 7 shows a modification of the liquid/blood transfusion system.

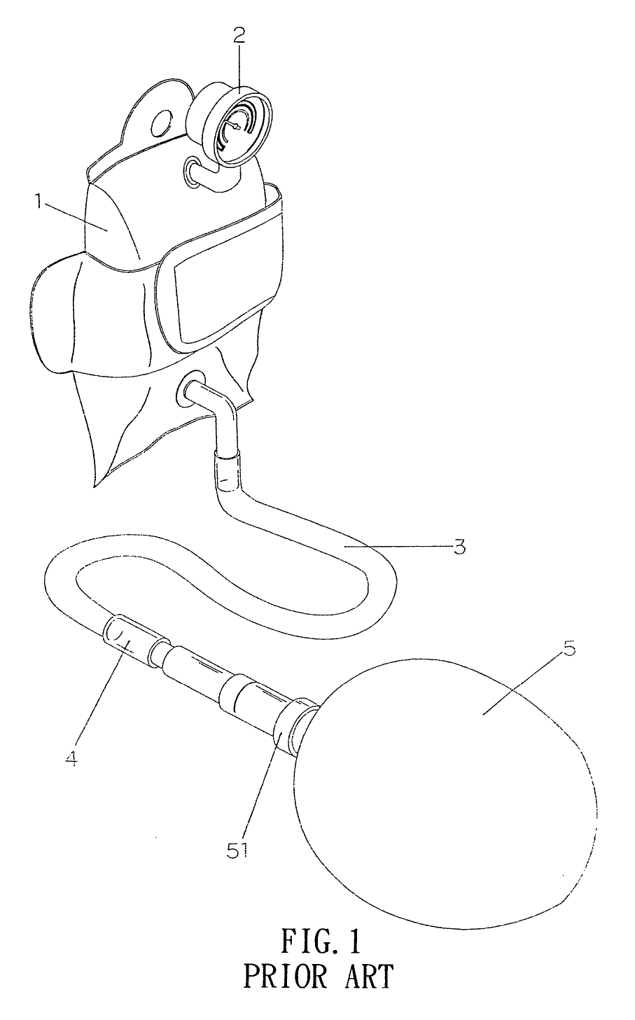

### DETAILED DESCRIPTION OF THE INVENTION

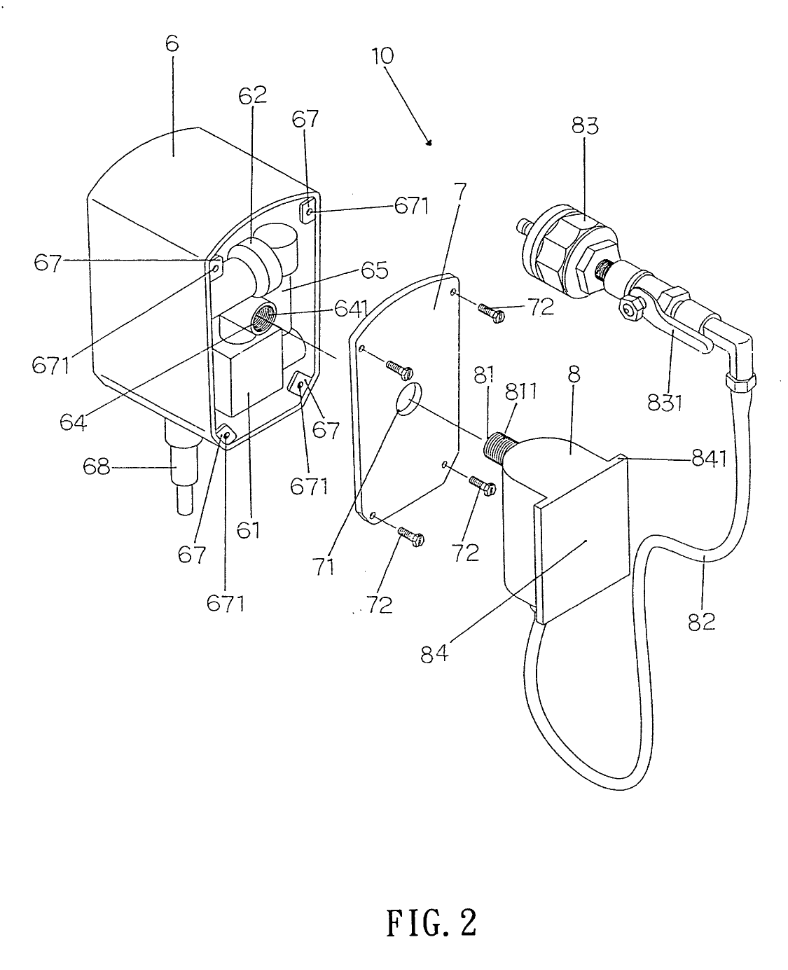

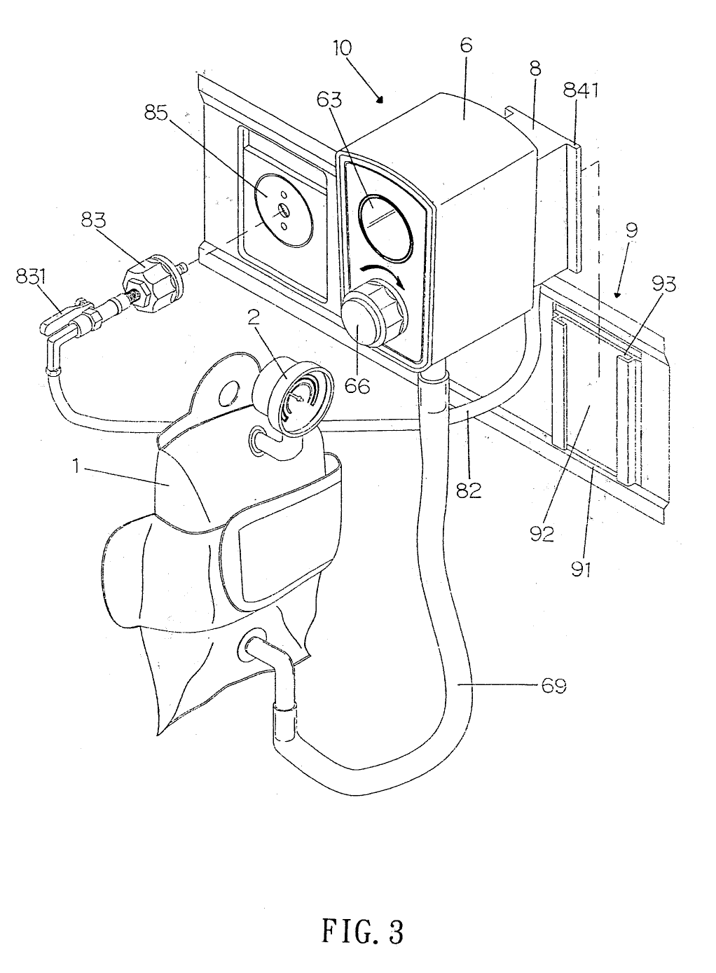

Referring now to the drawings wherein the showings are for purpose of illustrating preferred embodiments of the present invention only, and not for purposes of limiting the same. Referring to FIG. 2, an exploded view of a pressurization device for a transfusion system is illustrated, and in FIG. 3, a perspective view of an automatic pressurized transfusion system using the pressurization device is illustrated according to the present invention.

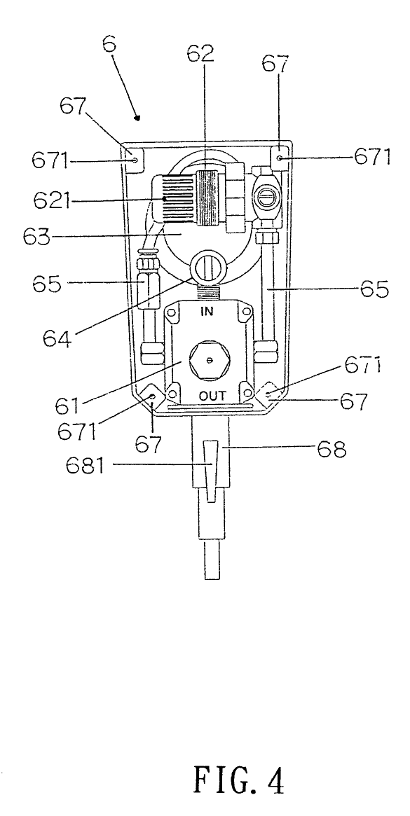

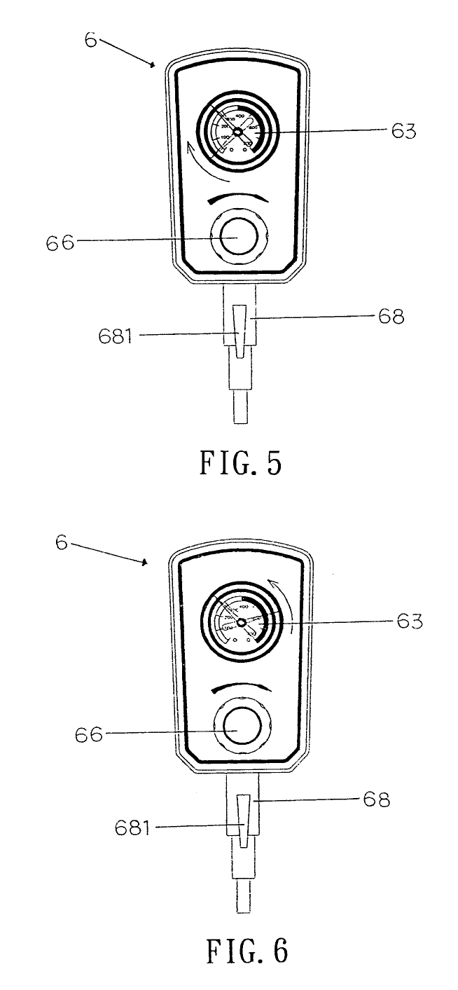

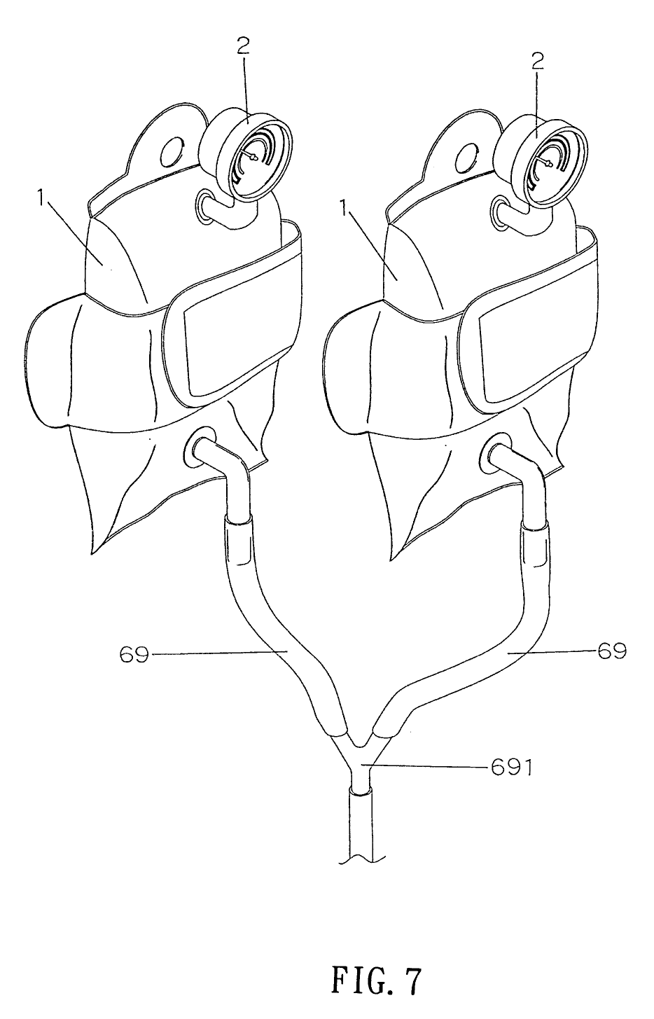

As shown in FIGS. 2 and 3, the pressurization device includes an enclosure 6, a lid 7, a gas reservoir 8, and a mount structure 9. FIG. 4 illustrates the rear panel pressurization device as shown in FIG. 2. The detailed structures of the pressurization device can be referred to FIGS. 2, 3 and 4.

As shown in FIG. 2, the enclosure 6 has a front panel, a sidewall, and open rear side to be covered with the lid 7. Inside of the enclosure 6, a valve 61, a pressure regulator 62, a pressure gauge 63, and a gas inlet 64 are installed. The gas inlet 64 opens at the open rear side to be connected to a pressurized gas supply. One end of the valve 61 is in fluid communication with the gas inlet 64, and the other end of the valve 61 is in fluid communication with a gas outlet 68. The gas outlet 68 is further connected to a gas delivery tube 69, such that the pressurized gas can be delivered to the liquid transfusion bag 1 connected at the other end of the gas delivery tube 69. Thereby, the liquid or blood contained in the liquid transfusion bag 1 can be pressurized by the gas to flow to the patient. The pressure gauge 64 is also in fluid communication with the gas outlet 68, such that the pressure applied to the liquid transfusion bag 1 can be monitored by the pressure gauge 63; and consequently, the flow rate of the liquid or blood transfused to the patient can be calculated and controlled in the real time. As shown in FIG. 2, the pressure gauge 63 is preferably installed and accessible on the front panel of the enclosure 6. An adjustment knob 66 in mechanical communication with the valve 61 is also installed and accessible on the front panel of the enclosure 6. The adjustment knob 66 controls the opening status of the valve 61, so as to control the pressure applied to the liquid transfusion bag 1. The enclosure 6 further comprises a plurality of lugs 67 extending along the open rear side, and each of the lugs 67 is perforated with an aperture 671. The lid 7 is also perforated with a plurality of holes along a periphery thereof. Therefore, by aligning the apertures 671 with the holes along the periphery of the lid 7, the lid 7 can be attached to the enclosure 6 using a plurality of fasteners such as screws 72. The lid 7 also includes an opening 71 to be aligned with the gas inlet 64, such that when the enclosure 6 is covered with the lid 7, the gas inlet 64 is open at the opening 71.

The gas reservoir 8 is preferably a rigid hollow body. In the embodiment as shown in FIG. 2, the gas reservoir 8 is in the form of a semi-cylindrical shell with a flat rear panel 84 serving as a fitting member to slide within the wall mount 9 as shown in FIG. 3. The gas reservoir 8 further includes a gas outlet 81 in the form of a male connector. Preferably, the gas inlet 61 of the enclosure 6 is in the form of a female connector, such that the gas reservoir 8 can be attached to the enclosure 6 is to be by fastening the gas inlet 61 with the gas outlet 81. In this embodiment, the interior sidewall of the gas inlet 61 and the exterior sidewall of the male connector 81 both include threads 641 and 811 formed thereon, respectively. The gas reservoir 8 further comprises a gas inlet extending from a bottom surface thereof. As shown in FIG. 3, the gas inlet is connected to the proximal end of a gas supply tube 82. The distal end of the gas supply tube 82 is connected with a plug 83 to be plugged into a gas outlet 85 of a gas supply source. Further, as shown in FIG. 2, two lateral sides of the flat rear panel 84 extend over the cylindrical shell to serve as two fitting slides 841 to slide through the slide channels 93 of the wall mount 9.

As mentioned above, the wall mount 9 includes a pair of slide channels 93 allowing the fitting slides 841 to be slide through. To avoid the whole gas reservoir 8 to slide through the wall mount 9, an elongate bottom rim 91 is formed to extend between the slide channels 93. Two opposing ends of the bottom rim 91 are adjacent to two bottom edges of the slide channels 93.

The pressure regulator 62 further includes a turning knob 621 at one end thereof for adjusting the gas leakage ratio. An independent pressure meter 2 is installed on top of the gas transfusion bag 1. The pressure meter 2 is in communication with the gas supply source of the pressurization device, such that the reading of the pressure meter 2 is the same as that read from the pressure gauge 63. The care giver or the user can thus monitors the gas pressure applied to the liquid transfusion bag 1 by either the pressure meter 2 or the pressure gauge 63. A valve switch 681 is further installed in the gas outlet 68 underneath the gas valve 64 to open or close the supply of the pressurized gas.

FIG. 7 shows a branching device 691 such as a Y-piece connected to a distal end of the gas delivery tube 69, such that the pressurization device is operative to provide pressurized gas to two liquid transfusion bags 1 simultaneously. Other configuration of the branching device 691 such as a fork-like structure which branches the gas delivery tube 69 into a plurality of paths can also be implemented without exceeding the spirit and scope of the present invention. Further, a gas source switch 831 is also installed at the distal end of the gas supply tube 82 to switch on and off the gas flow.

According to the above, to apply the automatic pressurized gas transfusion system provided by the present invention, the pressurization device is disposed in the wall mount 9 and connected to the liquid transfusion bag 1. A bag containing transfusion liquid is then wrapped within the liquid transfusion bag 1. The liquid transfusion bag 1 is then hung on a pole or a frame (not shown). The plug 83 is then plugged into the gas outlet 85 of the gas supply source, and the gas source switch 831 is switched open, such that gas supplied from the gas supply source flows from the gas reservoir 8 into the enclosure 6. Through the valve 61, the gas further flows to the liquid transfusion bag 1 through the gas delivery tube 69. The required pressure of the gas flow set up by the turning knob 621 of the pressurization device 62. When the gas pressure is insufficient, for example, lower than 300 mmHg as set up by the turning knob 621, the pressure regulator 62 automatically compensates the gas pressure to 300 mmHg. On the contrary, when the gas pressure is over 300 mmHg, the pressure within the pressurization device is automatically released by the pressure regulator 62 down to 300 mmHg.

Therefore, the automatic liquid transfusion system provided by the present invention provides a constant pressure to the gas transfusion bad, such that the transfusion liquid is supplied by a constant flow rate. The safety of the patient is thus ensured. Further, the volume of the system is relatively small compared to the conventional system. Further, the pressurization device can be mounted on the wall and easily removed therefrom, such that it is easily accessible by the medical practitioner or the care giver. Further, instead of frequent attachment and detachment of the pressurization device and the liquid transfusion bag, a switch is installed to facilitate the on/off operation of the fluid communication. Therefore, the lifetime of the system is prolonged.

This disclosure provides exemplary embodiments of the present invention. The scope of this disclosure is not limited by these exemplary embodiments. Numerous variations, whether explicitly provided for by the specification or implied by the specification, such as variations in shape, structure, dimension, type of material or manufacturing process may be implemented by one of skill in the art in view of this disclosure.

1. An automatic liquid or blood transfusion system, comprising:
a pressurization device including an enclosure having a front panel and an open rear end, the pressurization device further comprising the following devices installed in the enclosure:
a gas inlet from which a gas is supplied;
a valve having one end in fluid communication with the gas inlet;
a gas outlet in fluid communication with the other one end of the valve and at least one liquid transfusion bag;
a pressure gauge installed at the gas outlet to monitor pressure of the gas flowing through the gas outlet;
a pressure regulator in fluid communication with the gas outlet to regulate pressure of the gas flowing through the gas outlet at a constant value;
a control knob installed at the front panel of the enclosure to set up the constant value;
a lid, to cover the rear open end of the enclosure, wherein the lid is perforated with a central opening allowing the gas outlet open at the rear end of the enclosure;
a gas reservoir having a gas outlet to be connected with the gas inlet of the pressurization device, a gas inlet to be connected to a gas supply source, and a flat rear panel with two lateral protruding sides; and
a wall mount, having a pair of slide channels through which the protruding sides of the gas reservoir slide in, and a bottom rim extending between two bottom ends of the slide channels to hold the gas reservoir.

2. The system of claim 1, further comprising a switch installed at the gas outlet of the pressurization device.

3. The system of claim 1, wherein the gas reservoir comprises a hollow shell.

4. The system of claim 1, further comprising the gas delivery tube with one proximal end connected to the gas outlet of the pressurization device and one distal end connected to the liquid transfusion bag.

5. The system of claim 4, further comprising a branching device connected to the distal end of the gas delivery tube, such that the pressurization device can be connected to more than one liquid transfusion bag.

6. The system of claim 5, wherein the branching device includes a Y-piece.

7. The system of claim 1, further comprising a gas supply tube having a proximal end connected to the gas inlet of the gas reservoir and a distal end connected to the gas supply source.

8. The system of claim 7, further comprising a plug installed at the distal end of the gas supply tube.

9. The system of claim 8, further comprising a switch installed at the plug.

10. The system of claim 1, further comprising a gas meter mounted on top of the gas transfusion bag and connected to the gas supply source.

11. The system of claim 1, further comprising a turning knob to adjust gas leakage ratio of the pressure regulator.
